(19) United States
(12) Patent Application Publication
(10) Pub. No.: US 2023/0146217 A1
(43) Pub. Date: May 11, 2023

(54) MULTIFUNCTIONAL PHYSICAL STORE SYSTEM WITH AUTOMATIC STERILIZATION AND DISINFECTION FUNCTIONS

(71) Applicant: PROVIDENCE UNIVERSITY, TAICHUNG CITY (TW)

(72) Inventors: KUO-LIANG CHEN, TAICHUNG CITY (TW); KAE-KUEN HU, TAICHUNG CITY (TW); TAI-LUNG CHANG, TAICHUNG CITY (TW); MAO-AN CHU, TAICHUNG CITY (TW); PEI-HSUAN CHEN, TAICHUNG CITY (TW); NIEN-TZU HUANG, TAICHUNG CITY (TW); YUN-HSIN CHEN, TAICHUNG CITY (TW); YU-SHAN KE, TAICHUNG CITY (TW); CAI-TING SONG, TAICHUNG CITY (TW); CHI-YU KI, TAICHUNG CITY (TW); BO-MIN LIN, TAICHUNG CITY (TW)

(21) Appl. No.: 17/520,741

(22) Filed: Nov. 8, 2021

(51) Int. Cl. G06Q 50/12

(52) U.S. Cl. CPC G06Q 50/12

(57) ABSTRACT

A multifunctional physical store system with automatic sterilization and disinfection functions includes a central process device, and a disinfection device, an order device, a sterilization device, and a serve device that are coupled with the central process device. The system is applied to a physical store. The disinfection device is applied for carrying out the disinfection operation on the environment of the physical store. The sterilization device is applied for carryout surface sterilization on the people entering the store. The order device and the serve device are applied for people to order and acquire meals. Therefore, the application of various devices facilitates a nearly dust-free and sterile environment in the store, lowering the possibility of virus or bacterial infection between people in the operation area and the customers.

### BACKGROUND OF THE INVENTION

### 1. Field of the Invention

The present invention relates to intelligent physical store system, and more particularly, to a multifunctional physical store system with automatic sterilization and disinfection functions.

### 2. Description of the Related Art

In a conventional physical store, the cleansing and disinfection operations are manually carried out by staff of the store. Also, meal related operations, such as ordering, charging, and serving are mostly completed by the interaction of the counter staff and the client.

However, narrow places or small items are easily ignored by the store staff. Thus, manual cleansing operation carried out by staff easily cause existence of cleansing blind spots, lowering the tidiness of the environment. Also, manual disinfection easily causes less optimal disinfection effect due to inaccurate dosage of disinfection solution or unevenness of solution spraying.

Furthermore, with the global outbreak of COVID-19, although some stores use transparent object as a simple partitioning device for lowering the interaction between the counter staff and the customer, some meal related operations are still manually served by the counter staff to the customer, failing to facilitate a complete isolation therebetween, so that potential infection risk still exists between counter staff and customers.

### SUMMARY OF THE INVENTION

To improve the issues above, a multifunctional physical store system with automatic sterilization and disinfection functions is provided. With the automatic disinfection device, the store is periodically and completely disinfected and cleansed. Also, the disinfection device is combined with the sterilization device for achieving a nearly dust-free and sterile environment.

For achieving the aforementioned objectives, a multifunctional physical store system with automatic sterilization and disinfection functions in accordance with an embodiment of the present invention comprises a central control process device, a disinfection device, an order device, a sterilization device, and a serve device. The disinfection device is coupled with the central control process device. The disinfection device comprises a first control process module, an air intake module, and an air outtake module. The air intake module and the air outtake module are coupled with the first control process module, which controls the air intake time of the air intake module and the air outtake time of the air outtake module, respectively. The order device is coupled with the central process device and provided with an order module and the receive module. The order module comprises a first communication module, and the receive module comprises a second communication module, wherein the first communication module and the second communication module are communicatively connected in a wireless transmission manner, such that the order module can transmit a meal data to the receive module. The sterilization device is coupled with the central process device and comprises a second control process module, a detection module, and a disinfection spray module. The detection module and the disinfection spray module are coupled with the second control process module. When the detection module detects a person in the sterilization device, the second control process module controls the disinfection spray module to carry out the disinfection and sterilization process on the person. The serve device is coupled with the central process device and comprises a third control process module, a disinfection module, a conveyor belt, and two partition doors. The disinfection module, the conveyor belt, and the two partition doors are electrically connected with the third control process module. The two partition doors are disposed on two ends of the conveyor belt. The third control process module controls the disinfection module to disinfect the package materials of the meals. The third control process module controls the conveying speed of the conveyor belt. The third control process module controls the opening and closing status of the two partition doors, such that the two partition doors do not open simultaneously.

In another embodiment, a route indication device is included. The route indication device is coupled with the central process device, and is able to guide the person to move according to a predetermined route after passing through the sterilization device.

In another embodiment, the present invention is applied to a physical store. The physical store comprises a work area, a customer area, and a counter. The counter is disposed between the work area and the customer area. The disinfection device is disposed on the ceiling of the work area and the customer area. The receive module is disposed in the work area. The order module, and the sterilization device, and the route indication device are disposed in the customer area. The serve device is disposed on the counter.

With such configuration, the present invention applies the automatic disinfection device to carry out a wholesome disinfection cleansing operation. Also, with the combination of the disinfection device and the sterilization device, the store is allowed to have a nearly dust-free and sterile environment.

Further, through the order device, the serve device, and the route indication device, the people in the customer area can complete ordering and obtaining the meal without the necessity of contacting the staff in the work area. Thus, the chance of direct contact between the staff in the work area and the people in the customer area is completely avoided, thereby greatly reducing the infection of virus or bacteria between the staff in the work area and the people in the customer area.

### BRIEF DESCRIPTION OF THE DRAWINGS

FIG. 1 is a block view of the multifunctional physical store system with automatic sterilization and disinfection functions in accordance with an embodiment of the present invention.

FIG. 2 is a block view of the disinfection device in accordance with an embodiment of the present invention.

FIG. 3 is a block view of the order device in accordance with an embodiment of the present invention.

FIG. 4 is a block view of the sterilization device in accordance with an embodiment of the present invention.

FIG. 5 is a block view of the serve device in accordance with an embodiment of the present invention.

FIG. 6 is a block view of the route indication device in accordance with an embodiment of the present invention.

FIG. 7 is a perspective schematic view of the multifunctional physical store system with automatic sterilization and disinfection functions in accordance with an embodiment of the present invention.

FIG. 8 is a top schematic view of the multifunctional physical store system with automatic sterilization and disinfection functions applied to a physical store.

### DETAILED DESCRIPTION OF THE INVENTION

The aforementioned and further advantages and features of the present invention will be understood by reference to the description of the preferred embodiment in conjunction with the accompanying drawings where the components are illustrated based on a proportion for explanation but not subject to the actual component proportion.

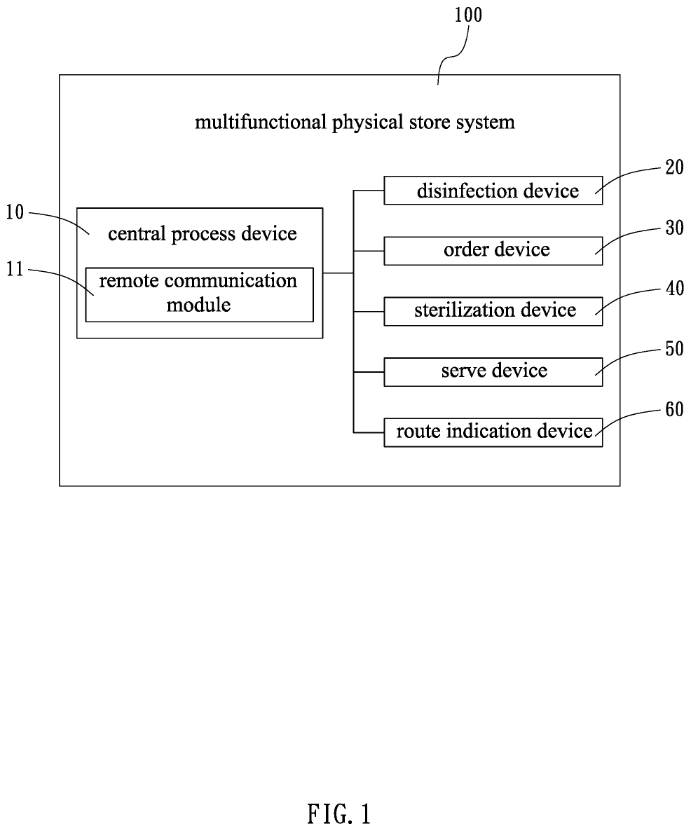

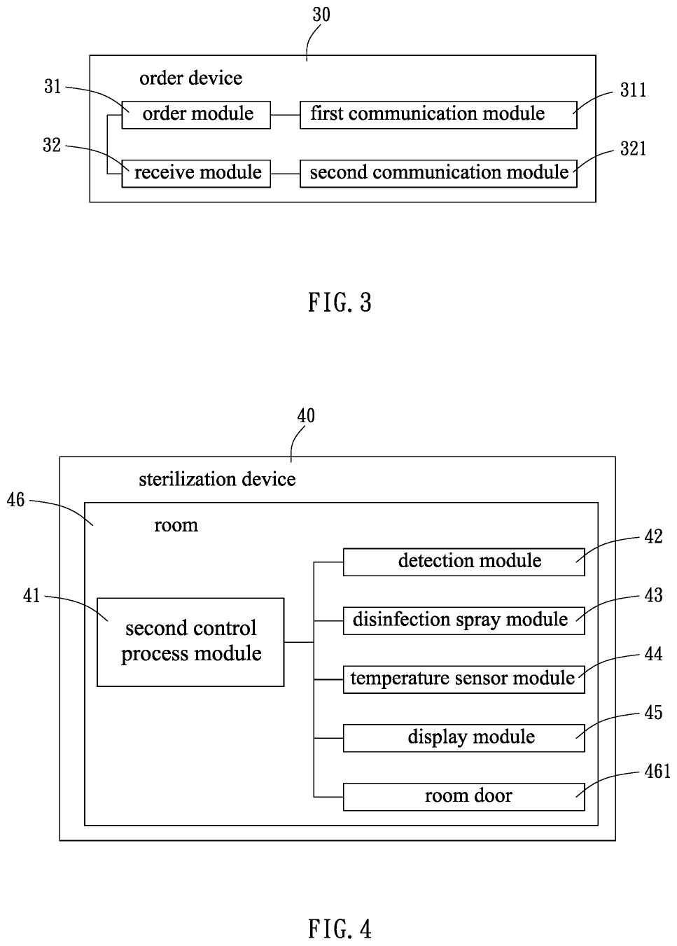

Referring to FIG. 1 to FIG. 8, a multifunctional physical store system 100 with automatic sterilization and disinfection functions in accordance with an embodiment of the present invention comprises a central process device 10, a disinfection device 20, an order device 30, a sterilization device 40, and a serve device 50. Therein, the present invention is applied to a physical store 1. The physical store 1 has an inner space 2, and the inner space 2 comprises a work area 2a, a customer area 2b, and a counter 2c. The customer area 2b comprises an entrance 2d and an exit 2e, wherein the entrance 2d is disposed on one side of the customer area 2b away from the work area 2a, and the exit 2e is disposed on another side of the customer area 2b away from the work area 2a and in opposite to the entrance 2d. The counter 2c is disposed between the work area 2a and the customer area 2b.

The central process device 10 is coupled with the disinfection device 20, the order device 30, the sterilization device 40, and the serve device 50. The central process device 10 is in the ceiling of the work area 2a and applied for controlling the operation of each device. Referring to FIG. 1, in the embodiment, the central process device 10 comprises a remote communication module 11, and the remote communication module 11 can be remotely connected with a terminal device (not shown) through the internet or wireless transmission. The terminal device remotely controls the operation of the disinfection device 20, the order device 30, the sterilization device 40, and the serve device 50 through the remote communication module 11.

The disinfection device 20 is coupled with the central process device 10 and disposed in the ceiling of the work area 2a and the customer area 2b. The disinfection device 20 comprises a first control process module 21, an air intake module 22, an air outtake module 23, a gas storage module 24, and a plurality of UV light sets 25 that are coupled with the first control process module 21. The first control process module 21 controls the air intake time of the air intake module 22, and controls the air outtake time of the air outtake module 23. The gas storage module 24 and the air intake module 22 are connected through a pipe. Therein, the disinfection device 20 further comprises a disinfection spray module 26 coupled with the first control process module 21. The first control process module 21 can periodically control the disinfection spray module 26 to spray disinfection liquid (such as but not limited to alcohol) for carrying out routine disinfection operations of the inner space 2.

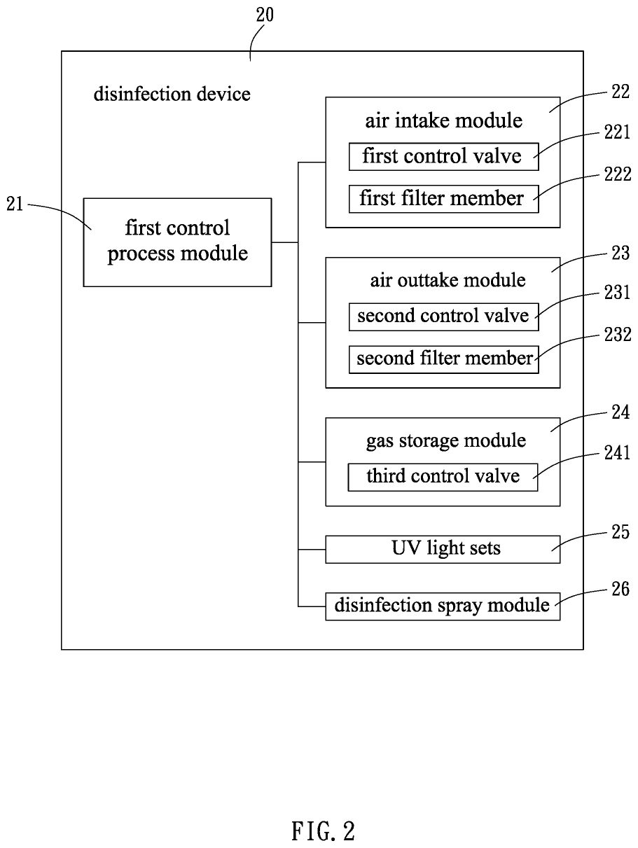

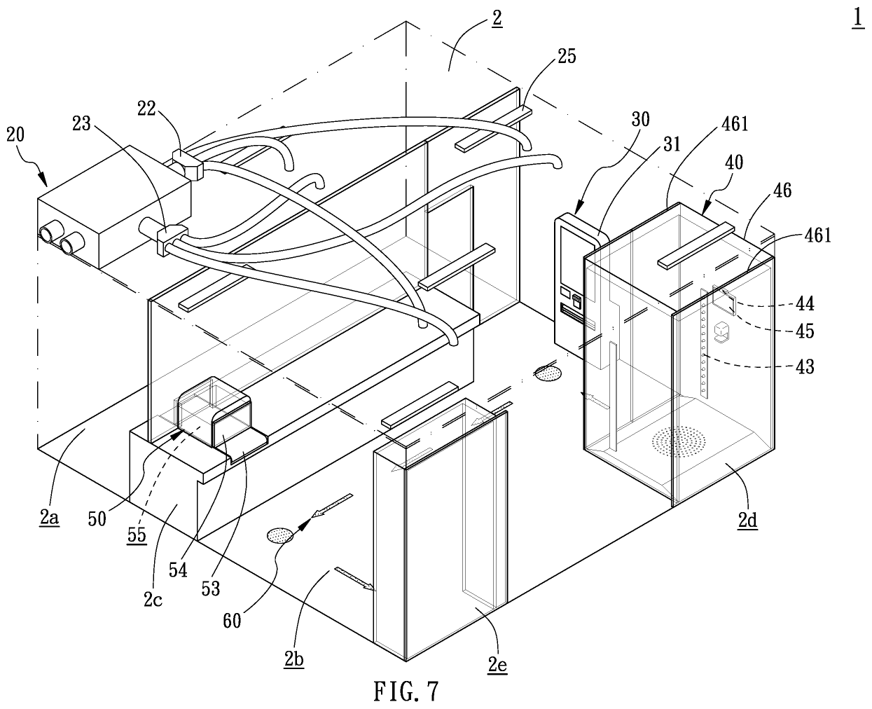

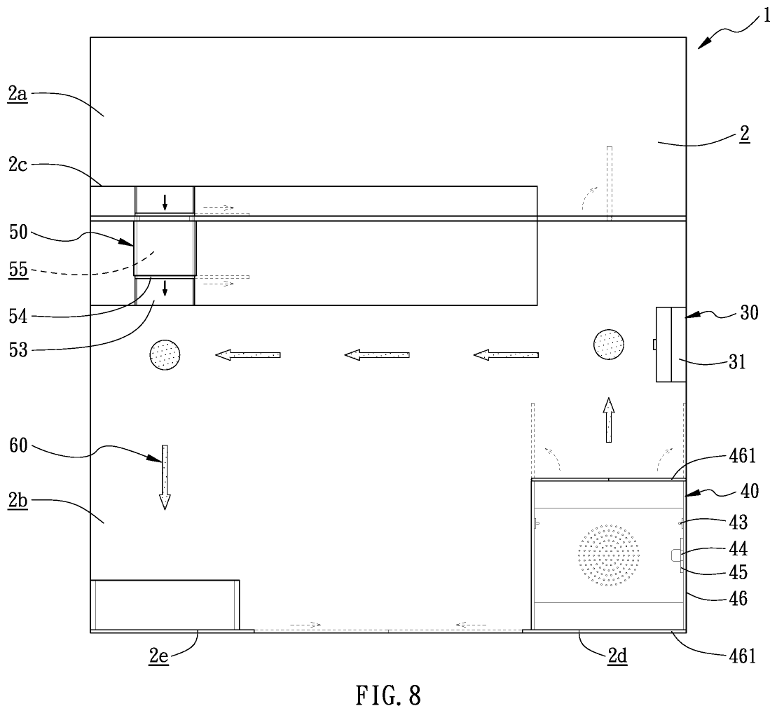

Referring to FIG. 2 in view of FIG. 7 and FIG. 8, in the embodiment, the air intake module 22 comprises a first control valve 221 and a first filter member 222. The air outtake module 23 comprises a second control valve 231 and a second filter member 232. Both the air intake module 22 and the air outtake module 23 have one end disposed outdoor and another end connected with the inner space 2, respectively. The gas storage module 24 comprises a third control valve 241.

When the physical store 1 is open, the first control process module 21 controls the first control valve 221 to open, so that the air intake module 22 filters outdoor air through the first filter member 222 and input the filtered air into the inner space 2. The first control process module 21 controls the second control valve 231 to open, so that the second filter member 232 of the air outtake module 23 filters and discharge the waste air in the inner space 2 out for cleansing the air in the inner space 2. When the physical store 1 is close, the first control process module 21 controls the first control valve 221 and the second control valve 231 to close. The first control process module 21 controls the third control valve 241 to open and turn on the plurality of UV light sets 25, such that the air intake module 22 inputs a disinfection gas (such as but not limited to ozone) which is stored in the gas storage module 24 for carrying out a comprehensive disinfection operation. Before the physical store 1 is open, the first control process module 21 controls the second control valve 231 to open, whereby the second filter member 232 of the air outtake module 23 filters and discharge the waste air in the inner space 2 out.

The order device 30 is coupled with the central process device 10 and comprises a serve module 31 and a receive module 32. The order module 31 is disposed in the customer area 2b and comprises a first communication module 311. The receive module 32 is disposed in the work area 2a and comprises a second communication module 321. The first communication module 311 and the second communication module 321 are communicatively connected through wireless transmission. When the person in the customer area 2b finishes the meal ordering process through the order module 31, the order module 31 can transmit a meal data through the first communication module 311 and the second communication module 321 to the receive module 32 for informing the staff in the work area 2a about the meal to be served. Through the remote ordering manner of the order module 31 and the receive module 32, the person in the customer area 2b can complete the meal ordering process without contacting the staff in the work area 2a.

The sterilization device 40 is coupled with the central process device 10 and disposed at the entrance 2d. The sterilization device 40 comprises a second control process module 41, a detection module 42, a disinfection spray module 43, a temperature sensor module 44, and a display module 45. The detection module 42, the disinfection spray module 43, the temperature sensor module 44 and the display module 45 are coupled with the second control process module 41. Referring to FIG. 4 in view of FIG. 7 and FIG. 8, in the embodiment, the sterilization device 40 comprises a room 46 having two room doors 461. The second control process module 41, the detection module 42, the disinfection spray module 43, the temperature sensor module 44, and the display module 45 are disposed in the room 46. The room doors 461 are coupled with the second control process module 41 and arranged on two sides of the room 46, respectively. The second control process module 41 controls the room doors 461 to optionally open.

When the detection module 42 detects a person in the room 46, the second control process module 41 shuts the room doors 461 and controls the disinfection spay module 43 to carry out the disinfection and sterilization process on the person. When the disinfection spray module 43 finished the disinfection and sterilization process, the second control process module 41 controls the temperature sensor module 44 to sense the body temperature of the person, and controls the display module 45 to display the body temperature of the person. Therefore, before the person enters the inner space 2, the virus and bacteria on the surface of the body and clothes of the person are cleansed. Therein, two room doors 461 are provided, wherein one room door 461 is disposed on one side of the room 46 near the entrance 2d, and the other room door 461 is disposed on another side of the room 46 away from the entrance 2d.

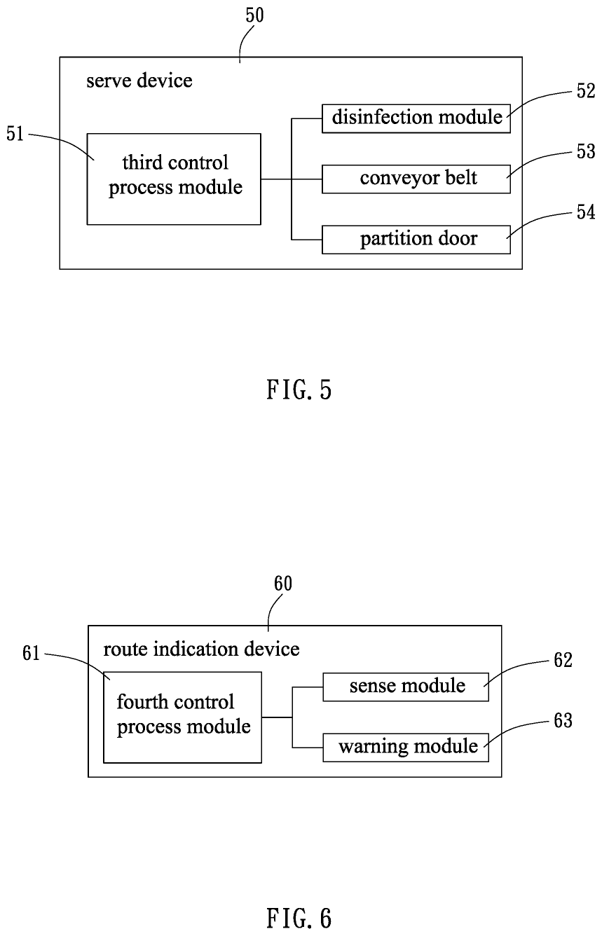

The serve device 50 is coupled with the central process device 10 and disposed on the counter 2c. The serve device 50 comprises a third control process module 51, a disinfection module 52, a conveyor belt 53, and two partition doors 54. The conveyor belt 53 and two partition doors 54 are electrically connected with the third control process module 51, respectively. The partition doors 54 are disposed on two ends of the disinfection module 52. The third control process module 51 controls the conveying speed of the conveyor belt 53 and the open and close statuses of the two partition doors 54, such that the two partition doors 54 do not open simultaneously. Referring to FIG. 5 in view of FIG. 7 and FIG. 8, in the embodiment, the serve device 50 comprises a disinfection space 55. The staff in the work area 2a place the meal on the convey belt 53 to be conveyed. The third control process module 51 controls the two partition doors 54 to close for sealing the disinfection space 55 in the close status, and then controls the disinfection module 52 to disinfect the package materials of the meal in the disinfection space 55. Therefore, in the meal serving and acquiring process between the people in the work area 2a and the customer area 2b, no direct contact is performed, so as to lower the virus or bacteria infection chance between the people in the work area 2a and the customer area 2b.

Referring to FIG. 1 and FIG. 6 in view of FIG. 7 and FIG. 8, in the embodiment, a route indication device 60 is further comprised. The route indication device 60 is coupled with the central process device 10 and disposed on the floor of the customer area 2b. The terminal device, through the remote communication module 11 of the central control process device 10, remotely controls the operation of the route indication device 60. The route indication device 60 comprises a fourth control process module 61, a sense module 62, and a warning module 63, wherein the sense module 62 and the warning module 63 are coupled with the fourth control process module 61, respectively. The route indication device 60 guides the person exiting the sterilization device 40 to orderly move along a predetermined route from the sterilization device 40 to the order module 31, the serve device 50, and the exit 2e. When the sense module 62 senses a person in the customer area 2b deviating from the predetermined route, the fourth control process module 61 controls the warning module 63 to send out warning. Thus, the route indication device 60 assures that the person in the customer area 2b orderly completing the meal ordering and acquiring process. Therein, the warning module 63 is, for example but not limited to, a warning light in the embodiment.

With the automatic disinfection device 20, the present invention periodically carries out a comprehensive disinfection cleansing operation. Also, the disinfection device 20 is combined with the sterilization device 40, such that the store can be provided with a nearly sterile environment.

Also, with the order device 30, and serve device 50, and the route indication device 60, the person in the customer area 2b can finish the meal ordering and acquiring process without contacting the staff in the work area 2a, thereby completely avoiding the direct contact between the people in the work area 2a and the customer area 2b, so that the virus or bacteria infection chance between the people in the work area 2a and the customer area 2b is greatly reduced.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A multifunctional physical store system with automatic sterilization and disinfection functions, comprising:
a central process device;
a disinfection device coupled with the central process device; the disinfection device comprising a control process module, an air intake module, and an air outtake module; the air intake module and the air outtake module coupled with the first control process module; the first control process module controlling an air intake time of the air intake module and an air outtake module of the air outtake module;
a order device coupled with the central process device; the order device comprising an order module and a receive module; the order module comprising a first communication module, and the receive module comprising a second communication module; the first communication module being communicatively connected with the second communication module through a wireless transmission, such that the order module transmits a meal data to the receive module;
a sterilization device coupled with the central process device; the sterilization device comprising a second control process module, a detection module, and a disinfection spray module; the detection module and the disinfection spray module being coupled with the second control process module; when the detection module detects a person in the sterilization device, the second control process module controls the disinfection spray module to carry out a disinfection and sterilization process on the person; and
a serve device coupled with the central process device; the serve device comprising a third control process module, a disinfection module, a conveyor belt, and two partition doors; the disinfection module, the conveyor belt, and the partition doors being electrically connected with the third control process module, respectively; the partition doors being disposed on two ends of the disinfection module; the third control process module controlling the disinfection module to disinfect a package material of meal; the third control process module controlling a conveying speed of the conveyor belt; the third control process module controlling an open and close status of the partition doors, such that the partition doors are prevented from opening simultaneously.

2. The multifunctional physical store system of claim 1, wherein the disinfection device comprises a gas storage module connected with the air intake module through a pipe; the first control process module controls the air intake module to filter outdoor air and input a filtered air or input a disinfection gas stored in the gas storage module into a store; the air outtake module discharges a waste air out from the store.

3. The multifunctional physical store system of claim 1, wherein the disinfection device comprises a plurality of UV light sets coupled with the first control process module; when a store is in the close status, the first control process module turns on the UV light sets to carry out a comprehensive disinfection and sterilization.

4. The multifunctional physical store system of claim 1, wherein the sterilization device comprises a temperature sensor module and a display module; the temperature sensor module and the display module are coupled with the second control process module; when the disinfection spray module finishes the disinfection and sterilization process, the second control process module controls the temperature sensor module to sense a body temperature of the person, and controls the display module to display the body temperature.

5. The multifunctional physical store system of claim 4, wherein a route indication device is further comprised; the route indication device is coupled with the central process device; the route indication device guides the person to move along a predetermined route after passing through the sterilization device.

6. The multifunctional physical store system of claim 5, wherein the route indication device comprises a fourth control process module, a sense module, and a warning module; the sense module and the warning module are coupled with the fourth control process module, respectively; when the sense module senses that the person deviates the predetermined route, the fourth control process module controls the warning module to send out a warning.

7. The multifunctional physical store system of claim 5, wherein the central process device comprises a remote communication module; the remote communication module is remotely connected with a terminal device through an internet or a wireless transmission; the terminal device remotely controls an operation of the disinfection device, the order device, the sterilization device, the serve device, and the route indication device through the remote communication module.

8. The multifunctional physical store system of claim 6, wherein the multifunctional physical store system with automatic sterilization and disinfection functions is applied to a physical store, wherein the physical store comprises a work area, a customer area, and a counter disposed between the work area and the customer area; the disinfection device is disposed in a ceiling of the customer area; the receive module is disposed in the work area; the order module, the sterilization device, and the route indication device are disposed in the customer area; the serve device in disposed on the counter.

9. The multifunctional physical store system of claim 8, wherein the customer area comprises an entrance and an exit; the entrance and the exit are disposed on two sides of the customer area away from the work area.

10. The multifunctional physical store system of claim 9, wherein the sterilization device is disposed at the entrance; the route indication device guides the person exiting the sterilization to orderly move along the predetermined route from the sterilization device to the order module, the serve device, and the exit.
